(19) United States
(12) Patent Application Publication
(10) Pub. No.: US 2021/0251552 A1
(43) Pub. Date: Aug. 19, 2021

(54) SYSTEM AND METHOD FOR RISK STRATIFICATION BASED ON DYNAMIC NONLINEAR ANALYSIS AND COMPARISON OF CARDIAC REPOLARIZATION WITH OTHER PHYSIOLOGICAL SIGNALS

(71) Applicant: THE JOHNS HOPKINS UNIVERSITY, Baltimore, MD (US)

(72) Inventors: Deeptankar DeMazumder, Baltimore, MD (US); Gordon Tomaselli, Lutherville, MD (US)

(21) Appl. No.: 17/233,208

(22) Filed: Apr. 16, 2021

Related U.S. Application Data
(63) Continuation of application No. 16/054,203, filed on Aug. 3, 2018, now abandoned.

(51) Int. Cl. A61B 5/364; A61B 5/316; A61B 5/349; A61B 5/352; A61B 5/369; A61B 5/00; A61B 5/0205

(52) U.S. Cl. CPC A61B 5/364; A61B 5/316; A61B 5/349; A61B 5/352; A61B 5/01; A61B 5/0006; A61B 5/02055; A61B 5/6898; A61B 5/369

(57) ABSTRACT

In accordance with an aspect of the present invention, a system and method allows for the assessment of health and mortality based on dynamic nonlinear calculations of self-similar fluctuation patterns in a time series of QT intervals and of other physiological signals, such as RR intervals, temperature, blood pressure, respiration, saturation of peripheral oxygen, intracardiac pressures, and electroencephalogram. In order to nonlinearly determine health and mortality, time series of QT intervals and of other physiological signals (e.g., RR intervals) are simultaneously obtained, and entropy values are calculated for each signal over the same temporal interval. “EntropyX” is calculated from relative changes between moments and entropy of QT intervals and those of other physiological signals over seconds to days. The absolute and relative entropy values at a specific time point and/or subsequent changes in entropy over future time points can be used to determine a treatment plan for the subject.

### CROSS REFERENCE TO RELATED APPLICATIONS

This application is a Continuation of U.S. patent application Ser. No. 16/054,203 filed Aug. 3, 2018, which is a Continuation of U.S. patent application Ser. No. 14/400,409, filed Nov. 11, 2014, which is a 35 U.S.C. § 371 U.S. national entry of International Application PCT/US2013/040751, having an international filing date of May 13, 2013, which claims the benefit of U.S. Provisional Application No. 61/646,830, filed May 11, 2012, and U.S. Provisional Patent Application No. 61/703,698 filed on Sep. 20, 2012, the content of each of the aforementioned applications is herein incorporated by reference in their entirety.

### GOVERNMENT SPONSORSHIP

This invention was made with government support under grant number HL091062, awarded by the National Institutes of Health. The government has certain rights in the invention.

### FIELD OF THE INVENTION

The present invention relates generally to cardiology. More particularly, the present invention relates to the dynamic nonlinear analyses of cardiac rhythm and of time-varying physiological signals to predict morbidity and mortality.

### BACKGROUND OF THE INVENTION

Electrocardiograms (ECGs) have long been studied in order to analyze cardiac function and predict health, disease and mortality. In many cases, linear deterministic methods in the time and frequency domains are used to analyze the information from the electrocardiogram. One such linear method, is referred to as heart rate variability (HRV). In time domain analyses, a range of normal values for HRV analyzed in the time domain, frequency domain and geometrically are established based on 24-hour ambulatory recordings. Similar metrics, particularly in the time domain, are not universally accepted for short-term recording so stratification of continuous data can be used.

In contrast to time domain analyses, that do little to account for irregularities, the irregularity in the time-sampled intervals of electrocardiographic ventricular activation (RR) and repolarization (QT) have been accounted for in frequency domain analyses in order to calculate an estimate of the power spectrum density (PSD). Because the typical PSD estimators implicitly assume equidistant sampling, they cannot be directly applied to the RR and QT interval time series because it is their variability that the method is trying to quantify. Therefore, the interval time series is first converted to a time series with equidistant sampling using, for example, a cubic spline interpolation method to avoid generating additional harmonic components in the spectrum. Other methods for equidistant sampling conversion include interpolation based on weighted average of recent intervals and the Lomb periodogram.

The typical methods employed for PSD estimation include the fast Fourier transform (FFT) and autoregressive (AR) models. In the FFT method, spectrum powers are calculated by integrating the spectrum over the frequency bands. In contrast, the parametric AR method models the time series as a linear combination of complex harmonic functions, which include pure sinusoids and real exponentials as special cases, and fits a function of frequency with a predefined number of poles (frequencies of infinite density) to the spectrum. The AR method asserts that the position and shape of a spectral peak is determined by the corresponding complex frequency and that the height of the spectral peak contains little information about the complex amplitude of the complex harmonic functions. In the AR method, the spectrum is divided into components and the band powers are obtained as powers of these components.

There are several fundamental limitations to all forms of frequency domain analyses. Nonstationarity in time series severely limits the range of frequencies that can be studied by all methods of frequency-domain analyses. Frequency-domain analyses, while retaining some information relating to ordering of observations, conceal details of interactions between mechanisms (e.g., respiration-mediated change in heart rate may stimulate other mechanisms). Heart rates have self-similar fluctuations, affected not only by the most recent value but also by much more remote events, or in other words, a “memory” effect. In time series, these phenomena may be quantified as a repetitive pattern of fluctuation, but in the frequency domain, it may be indistinguishable from uncorrelated fluctuations. Although AR models may provide better resolution in shorter time series than FFT analysis, they include assumptions about model complexity, contingency of negative components in spectral factorization and discard information in the input time series (i.e., reduced degrees of freedom).

Nonlinear dynamic analyses are an alternate approach for understanding the complexity of biological systems. By definition, a nonlinear system has an output that is simply “not linear,” i.e., any information that fails criteria for linearity⁴, output proportional to input and superposition, behavior predicted by dissecting out individual input-output relationships of sub-components.

Virtually all biological signals demonstrate nonlinear properties. A simple common example is nonstationarity (e.g., drift in heart rate or blood pressure during sleep-wake cycles). Although a variety of stationarity tests provide useful measures, some arbitrary criteria are needed to judge stationarity and as such, important information on pathological states and natural physiological processes contained within these nonstationary properties are lost, as illustrated in FIGS. 1A-1C. FIG. 1A illustrates fractal temporal processes of a healthy RR interval time series; FIG. 1B illustrates wavelet analysis of healthy RR time series of >1500 beats (x-axis is time, y-axis is wavelet scale (5 to 300 secs); FIG. 1C illustrates the wavelet amplitudes.

It is quite common for the output of nonlinearly coupled control systems to generate behaviors that defy explanation based on conventional linear models. Characteristic behaviors of nonlinear systems include self-sustained, periodic waves (e.g., ventricular tachycardia), abrupt changes in output (e.g., sudden onset of ventricular fibrillation) and, possibly, chaos.

On the other hand, nonlinear systems that appear to be very different in their specific details may exhibit certain common output patterns, a characteristic referred to as universality. Moreover, outputs may change in a sudden, discontinuous fashion (e.g., bifurcation), often resulting from a very small change in one of the control modules. For example, the same system may produce a wildly irregular output that becomes highly periodic or vice versa, e.g., electrical alternans, ST-T wave alternans preceding ventricular fibrillation, pulsus alternans during congestive heart failure.

Prior studies have used various nonlinear measures of RR interval complexity, including Poincaré plot, various forms of entropy analysis, and detrended fluctuation analysis to provide insight into heart rate regulatory mechanisms and prediction of adverse events. The following sections describe the ECG analysis strategies employed in the Hopkins PROSe study of patients with an implantable cardioverter-defibrillator (ICD) (clinical trial registration #NCT00733590) and in the Sleep Heart Health study, including novel nonlinear strategies for quantifying the dynamics of QT interval time series and for nonlinearly comparing the QT interval time series with the RR interval time series and other time-varying physiological signals.

The Poincare plot is a graphical representation of the correlation between successive RR intervals, i.e. plot of RRₙ₊₁ as a function of RRₙ. The significance of this plot is that it is the two-dimensional reconstructed phase space, i.e., the projection of the system attractor that describes the dynamics of the time series. Because an essential feature of this analysis method is the shape of the plot, prior studies have parameterized the shape to fit an ellipse oriented according to the line-of-identity, e.g., for a first order plot, RRₙ=RRₙ₊₁. A cigar-shaped plot along the principal diagonal (x=y) would reveal high autocorrelation within the time series and a circular plot would reveal periodicity, e.g., the Poincaré plot of a sine wave or a pendulum is a circle. Because Poincare plots are based on linear statistics', they do not capture the nonlinear temporal dynamics of the time series.

Detrended fluctuation analysis (DFA) is a nonlinear strategy employed in prior studies for gaining insight into temporal dynamics and for mortality risk prediction by measuring correlations within the RR time series. Typically, the correlations are divided into short-term (α₁, range 4≤n≤16) and long-term (α₂, range 16≤n≤64) fluctuation [0<α<0.5 indicates a large value is followed by a small value and vice versa, 0.5<α<1.0 indicates a large value is likely to be followed by a large value]. An a value of 0.5, 1.0, >1.0, or >1.5 indicates white noise, 1/f noise, different kinds of noise, or brown noise (integral of white noise), respectively.

Classical information theory, founded by Claude Shannon has been widely utilized for the study of nonlinear signals. Related to thermodynamic entropy, the information entropy can be calculated for any probability distribution (i.e., occurrence of an event that had a probability of occurring out of the space of possible events). The information entropy quantifies the amount of information needed to define the detailed microscopic state of a system, given its macroscopic description, and can be converted into its thermodynamic counterpart based on the Boltzmann distribution. Recent experimental evidence supports this method of conversion.

Shannon entropy (ShanEn) measures information as the decrease of uncertainty at a receiver (or physiological process). ShanEn of the line length distribution is defined as

$$ShanEn = {\sum }_{{l}_{min}}^{{l}_{max}} {n}_{l} ln {n}_{l}$$

where nₗ is the number of length l lines such that

$${n}_{l} = \frac{{N}_{l}}{{\sum }_{{l}_{min}}^{{l}_{max}} {N}_{{l}^{′}}}$$

From a chemical thermodynamics perspective, the reduced AG would be equal to the minimum number of yes/no questions (using loge) that needed to be answered in order to fully specify the microscopic state, given the macroscopic state. An increase in Shannon entropy indicates loss of information.

For clinical application to short and noisy time series, another measure “approximate entropy” (ApEn) was developed based on the Kolmogorov entropy, which is the rate of generation of new information. ApEn examines time series for similar epochs such that the presence of more frequent and more similar epochs, i.e., a high degree of regularity, lead to lower ApEn values.

A related method but much more accurate than ShanEn or ApEn is Sample entropy (SampEn), which unlike ApEn, does not count self-matches of templates, does not employ a template-wise strategy for calculating probability and is more reliable for shorter time series. SampEn is the conditional probability that that two short templates of length m that match within a tolerance r (where r=0.2×standard deviation of the signal) will continue to match at the next point m+1.

SampEn is calculated by first forming a set of vectors uⱼ of length m

uⱼ=(RRⱼ,RRⱼ₊₁, . . . ,RRⱼ₊ₘ₋₁),j=1,2, . . . N−m+1

where m represents the embedding dimension and N is the number of measured RR intervals. The distance between these vectors is defined as the maximum absolute difference between the corresponding elements

d(uⱼ,uₖ)=max[|RRⱼ₊ₙ−RRₖ₊ₙ∥n=0, . . . ,m−1]

For each uⱼ, the relative number of vectors uₖ for which d(uₚ,uₖ)≤r is calculated as

$${C}_{j}^{m} ( r ) = \frac{number of [ {u}_{k} \vert d ( {u}_{j} , {u}_{k} ) \leq r ]}{N - m + 1} ∀ k \neq j$$

with values of Cⱼ^{m}(r) ranging between 0 and 1. Average of Cⱼ^{m}(r) yields

$${C}^{m} ( r ) = \frac{1}{N - m + 1} {\sum }_{j = 1}^{N - m + 1} {C}_{j}^{m} ( r )$$

$$and$$

$$SampEn ( m , r , N ) = - ln \frac{{C}^{m + 1} ( r )}{{C}^{m} ( r )}$$

Although the development of SampEn was a major advancement in application of information theory to heart rate dynamics, SampEn has a few significant limitations. What is the optimal value of m? How does one pick r? The usual suggestion is that m should be 1 or 2, noting that there are more template matches and thus less bias for m=1, but that m=2 reveals more of the dynamics of the data. The convention has been that m=2 and r=0.2×standard deviation of the epoch, and these criteria were set on empirical grounds.

The coefficient of sample entropy (COSEn), an optimized form of the SampEn measure, was originally designed and developed at the University of Virginia using m=1 for the specific purpose of discriminating atrial fibrillation from normal sinus rhythm (NSR) from surface ECGs at all heart rates using very short time series of RR intervals, i.e., about 12 heart beats. As with ApEn and SampEn, smaller values of COSEn indicate a greater likelihood that similar patterns of RR fluctuation will be followed by additional similar measurements. If the time series is highly irregular, the occurrence of similar patterns will not be predictive for the following RR fluctuations and the COSEn value will be relatively large.

Using the same parameters [i.e., length of template or embedding dimension (m)=1], COSEn was subsequently optimized for analysis of intracardiac electrograms (EGMs) and validated in the Johns Hopkins PROSe-ICD study, requiring only 9 RR intervals before ICD shock to accurately distinguish atrial fibrillation from lethal ventricular arrhythmias and outperforming representative discrimination algorithms used in contemporary ICDs for therapy (DeMazumder et al. Circulation Arrhythmia and Electrophysiology, in press).

Because nonlinear metrics have better discrimination ability than other conventional methods and time-varying physiological signals such as the cardiac rhythm have been shown to reflect health and disease, it would therefore be advantageous to provide a more accurate method for nonlinearly quantifying the dynamics of the RR, QT and other time-varying physiological signals for prediction of health and mortality.

### SUMMARY OF THE INVENTION

The foregoing needs are met, to a great extent, by the present invention, wherein in one aspect a method of nonlinearly determining health and mortality includes obtaining a ventricular repolarization interval (QT) time series from a subject for a temporal interval and obtaining a ventricular activation interval (RR) time series from the subject for the same temporal interval. The method includes first, calculating entropy in the QT time series over the temporal interval to determine health and mortality. The method also includes calculating additional entropy values over the same temporal interval for the RR and other time-varying physiological signals such as the temperature, blood pressure, respiration, saturation of peripheral oxygen, intracardiac pressures and electroencephalogram time series. Additionally, the method includes comparing the first QT entropy with the entropy values of the other physiological signals to determine health and mortality.

The absolute baseline entropy value provides information regarding health and mortality risk. Moreover, relative changes in entropy over a subject's follow up period provide dynamic information regarding health and mortality risk. The determination of health and mortality can then be used to create a treatment plan for the subject. The computing device can also include a comparison of the first QT entropy with the other entropies to determine health and mortality using the equations listed below.

The general form of the equation for calculating the entropy from the time series of a physiological signal (Y) is:

$${EntropyX}_{Y} ( m , N , n , R ) = E n t r o p y {X}_{α} - [ - ln \frac{{C}_{Y}^{m + 1} ( {r}_{Y} )}{{C}_{Y}^{m} ( {r}_{Y} )} + ln ( 2 \times {r}_{Y} ) ] \qquad ( Equation 1 )$$

where the embedding dimension or template length (m)≥3, the number of sampled intervals per bin of the time series (N) is ≥20, the sufficient number of matches n≥(N÷5), r is the calculated tolerance for a given N that satisfies the specified n but without perfect matches, R represents the specified precision of the data from which the initial value of the tolerance r is designated for subsequent iterative calculations, C_{Y}^{m}(r_{Y}) represents the total number of matches within r of length m in the Y time series, C_{Y}^{m+1}(r_{Y}) represents the total number of matches within r of length m+1 in the Y time series, and EntropyX_{α} represents the entropy of the time series of another physiological signal such as the QT, RR, temperature, blood pressure, respiration, intracardiac pressures, saturation of peripheral oxygen or electroencephalogram time series.
The value of r is an important factor for determining the underlying dynamics of a segment of intervals. If r is too small (i.e., smaller than the typical noise amplitude), then a group of m intervals that are similar shall fail to match. However, if r is too large, there will be a loss in discriminating power simply because the group of intervals will look similar to one another given sufficiently lax matching conditions. The ideal condition would be to vary r with the scale of signal noise such that r is as small as possible for searching for order in the dynamics while ensuring the number of matches remains large enough to ensure precise statistics. This is analogous to varying the bin widths of a histogram to optimally describe its distribution. Therefore, the calculation of equation 1 requires the following additional steps.
First, the iterative calculation of the tolerance (r) for each bin of time series data (N) is determined by first calculating the initial value of r by the equation:

r=R×[k+0.5] (Equation 2)

where k=N÷R (k is rounded to next lowest integer) and the value of R is specified based on resolution of the time series data, e.g., values of R typically range between 1 and 5 for routine surface ECG measurements, values of R are typically ≤1 for intracardiac EGM recordings, and values of R may be ≥5 for noisy or interpolated ECG data.
For each iteration of Cⱼ^{m+1}(r), r is allowed to vary such that a sufficient number of matches [denoted by n≥(N÷5)] are found, albeit without using larger values of r than necessary for confident entropy estimation.
For example, if the value of r is too small to find sufficient matches, i.e., C^{m+1}(r)<n, then

k=k+x (Equation 3)

for calculation of a new value for r using equation 2 where x is a constant positive integer that is specified at the onset of the analysis based on the precision and scale of the acquired signal (e.g., x=0.5 for the typical RR time series), followed by reiteration of Cⱼ^{m+1}(r).

However, if the value of r is too large, i.e., C^{m+1}(r)=C^{m}, then

$$k = \frac{R {R}_{max} - R {R}_{min}}{R} \qquad ( Equation 4 )$$

for calculation of a new value for r using equation 2, followed by reiteration of Cⱼ^{m+1}(r).
When C^{m+1}(r)≥n and C^{m+1}(r)≠C^{m}, then the negative natural logarithm of the calculated conditional probability [i.e., C^{m+1}(r)≠C^{m}(r)] divided by the matching region area (2×r), i.e., −ln[{C^{m+1}(r)÷C^{m}(r)}÷(2×r)]=−ln{C^{m+1}(r)÷C^{m}(r)}+ln(2×r), is defined as EntropyX_{Y} for a single physiological signal (i.e., EntropyX_{α}=0).
For example, when the entropy of the QT interval is not compared to that of another signal, i.e., EntropyX_{α}=0, the equation for calculating the entropy of the QT interval time series is:

$${EntropyX}_{QT} ( m , N , n , R ) = [ - ln \frac{{C}_{Q T}^{m + 1} ( {r}_{Q T} )}{{C}_{Q T}^{m} ( {r}_{Q T} )} + ln ( 2 \times {r}_{Q T} ) ] \qquad ( Equation 5 )$$

Similarly, when the entropy of the RR interval is not compared to another signal, the equation for calculating the entropy of the RR interval time series is:

$${EntropyX}_{RR} ( m , N , n , R ) = [ - ln \frac{{C}_{RR}^{m + 1} ( {r}_{Q T} )}{{C}_{RR}^{m} ( {r}_{Q T} )} + ln ( 2 \times {r}_{RR} ) ] \qquad ( Equation 6 )$$

As with ApEn and SampEn, smaller values of EntropyX_{∝} indicate a greater likelihood that similar patterns of measurements will be followed by additional similar measurements. If the time series is highly irregular, the occurrence of similar patterns will not be predictive for the following measurements and the EntropyX_{∝} value will be relatively large.

For comparing the degree of dissimilarity in the dynamics of two time series, a and Y, each consisting of N number of intervals:

$$\begin{matrix}{EntropyX}_{α {Y}_{1}} ( {m}_{α} , {m}_{Y} , {r}_{α} , {r}_{Y} , N , n , {R}_{α} , {R}_{Y} ) = [ - ln \frac{{C}^{m + 1} ( {r}_{α} )}{{C}^{m} ( {r}_{α} )} + ln ( 2 \times {r}_{α} ) ] - [ - ln \frac{{C}^{m + 1} ( {r}_{Y} )}{{C}^{m} ( {r}_{Y} )} + ln ( 2 \times {r}_{Y} ) ] & ( Equation 7 ) \\ {EntropyX}_{α {Y}_{2}} ( {m}_{α} , {m}_{Y} , {r}_{α} , {r}_{Y} , N , n , {R}_{α} , {R}_{Y} ) = - \frac{{C}^{m + 1} ( {r}_{α} )}{{C}^{m} ( {r}_{α} )} \times ln \frac{{C}^{m + 1} ( {r}_{Y} )}{{C}^{m} ( {r}_{Y} )} + ln [ {r}_{Y} \times {r}_{α} ) & ( Equation 8 )\end{matrix}$$

For nonlinear comparison of the dynamics of the Y time series consisting of N number of intervals with the dynamics of the time series of another physiological signal (α) also consisting of N number of intervals, each time series is first normalized over its respective range of values and then, C_{Yα}^{m}(r_{Y}) is defined as the total number of matches in the Y time series within r_{Y} of templates formed in the a time series within r_{α} of length m, C_{Yα}^{m+1}(r_{Y}) is defined as the total number of matches in the Y time series within r_{Y} of templates formed in the a time series within r_{α} of length m+1, C_{αY}^{m}(r_{α}) is defined as the total number of matches in the a time series within r_{α} of templates formed in the Y time series within r_{Y} of length m, and C_{αY}^{m+1}(r_{α}) is defined as the total number of matches in the a time series within r_{α} of templates formed in the Y time series within r_{Y} of length m+1.

$$\begin{matrix}{EntropyX}_{α {Y}_{3}} ( {m}_{α} , {m}_{Y} , {r}_{α} , {r}_{Y} , N , n , {R}_{α} , {R}_{Y} ) = - ln \frac{{C}^{m + 1} ( {r}_{α} )}{{C}^{m} ( {r}_{Y} )} & ( Equation 9 ) \\ {EntropyX}_{α {Y}_{4}} ( {m}_{α} , {m}_{Y} , {r}_{α} , {r}_{Y} , N , n , {R}_{α} , {R}_{Y} ) = - ln \frac{{C}^{m + 1} ( {r}_{Y} )}{{C}^{m} ( {r}_{α} )} & ( Equation 10 ) \\ {EntropyX}_{α {Y}_{5}} ( {m}_{α} , {m}_{Y} , {r}_{α} , {r}_{Y} , N , n , {R}_{α} , {R}_{Y} ) = [ - ln \frac{{C}^{m + 1} ( {r}_{α} )}{{C}^{m} ( {r}_{α} )} ] - [ - ln \frac{{C}^{m + 1} ( {r}_{Y} )}{{C}^{m} ( {r}_{Y} )} ] + ln ( {r}_{Y} \times {r}_{α} ) & ( Equation 11 )\end{matrix}$$

The treatment plan created can include monitoring the subject's cardiac rhythms and other time-varying physiological signals, including but not limited to the QT interval, RR interval, temperature, blood pressure, respiration, saturation of peripheral oxygen, intracardiac pressures, and electroencephalogram. The subject can further be one selected from the group consisting of humans, primates, dogs, guinea pigs, rabbits, horses, cats, fruit flies and other organisms.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates fractal temporal processes of a healthy RR interval time series according to an embodiment of the present invention; FIG. 1B illustrates wavelet analysis of healthy RR time series of >1500 beats (x-axis is time, y-axis is wavelet scale (5 to 300 secs) according to an embodiment of the present invention; FIG. 1C illustrates the wavelet amplitudes according to an embodiment of the present invention.

FIGS. 2, 3, and 4 illustrate a time series of RR and QT intervals for heart failure patients alive after 37, 56, 88 months, respectively, of follow up, according to an embodiment of the present invention. For each figure, the time series in the top panel was divided into ten bins, each bin consisting of 30 consecutive intervals, and the first two bins are shown in the middle panel; the bottom panel shows a phase plot for the RR and QT interval time series and corresponding plots of the mutual information and total correlation between the RR and QT time series, according to an embodiment of the present invention.

FIGS. 5 and 6 illustrate a time series of RR and QT for patients who died from septic shock after 84 and 53 months of follow-up, respectively according to an embodiment of the present invention. For each figure, the time series in the top panel was divided into ten bins, each bin consisting of 30 consecutive intervals, and the first two bins are shown in the middle panel; the bottom panel shows a phase plot for the RR and QT interval time series and corresponding plots of the mutual information and total correlation between the RR and QT time series, according to an embodiment of the present invention.

FIG. 7 illustrates a three dimensional plot of the adjusted hazard ratios of EntropyX_{QT} (also referred to as EntropyX₀ or EnX₀) in heart failure patients (N=851) for association with sudden cardiac death (N=149) as a function of the embedding dimension (m) and the number of intervals in each bin or bin width (w); the peak hazard ratio occurred at around m=4 and w=40 according to an embodiment of the present invention. After normalization of all continuous variables, the hazard ratios of EntropyX₀ was adjusted for demographics (age at implant, gender, race), medical history (history of paroxysmal atrial fibrillation, smoking, hypertension, diabetes mellitus, ischemic cardiomyopathy), clinical exam (body mass index, NYHA class, mean arterial pressure), prescribed medications (aspirin, beta blocker, ACE inhibitor and/or ARB, aldosterone antagonist, statin, antiarrhythmics, loop diuretics), laboratory results (Na, K, BUN), biomarkers (hsCRP, proBNP), left ventricular ejection fraction, and linear ECG analyses (heart rate, percent premature ventricular contractions, heart rate variability, heart rate frequency domain analysis of LF:HF ratio, QT-heart rate coherence, and QT variability index), according to an embodiment of the present invention. From these sensitivity analyses in heart failure patients, the optimal m, N, n and R were determined to 4, 40, 8, and 1, respectively, according to an embodiment of the present invention.

FIG. 8 Effect of EntropyX_{QT} (also referred to as EntropyX₀ or EnX₀) by quintiles on incrementally adjusted proportional hazards ratio in models 1-4 in the Johns Hopkins PROSe ICD study, according to an embodiment of the present invention.

FIG. 9 Multivariate-adjusted hazard ratios from EntropyX_{QT} (also referred to as EntropyX₀ or EnX₀) in the Johns Hopkins PROSe ICD study for association with sudden cardiac death and all-cause mortality, according to an embodiment of the present invention.

FIG. 10 Table of risk prediction improvement with EntropyX_{QT} (also referred to as EntropyX₀ or EnX₀) in the Johns Hopkins PROSe ICD study, according to an embodiment of the present invention.

FIG. 11 Table of patient and ECG characteristics by quintiles of EntropyX_{QT} (also referred to as EntropyX₀ or EnX₀) in the Johns Hopkins PROSe ICD study, according to an embodiment of the present invention.

FIG. 12 Table of patient and ECG characteristics by events in the Johns Hopkins PROSe ICD study, according to an embodiment of the present invention.

FIG. 13 Comparison of receiver operating characteristic (ROC) curves between base and enhanced models in the Johns Hopkins PROSe ICD study, according to an embodiment of the present invention.

FIG. 14 Plots of stages of sleep, heart rate variability (SDNN_msec), EntropyX_{RR} (also referred to as RR entropy), frequency domain analyses of low frequency power (LFPow), high frequency power (HFPow) and percent low frequency power (% LF) in the Sleep Heart Health Study, according to an embodiment of the present invention.

FIG. 15 Plots of QT variability index (QTVI), EntropyX_{QT} (also referred to as EntropyX₀ or EnX₀), Bazett heart rate corrected QT interval (QTc), QT:RR correlation coefficient (QTRR r2), mean QT:RR coherence, and EntropyX_{RRQT1} (also referred to as EntropyX₁ or EnX₁) in the Sleep Heart Health Study, according to an embodiment of the present invention.

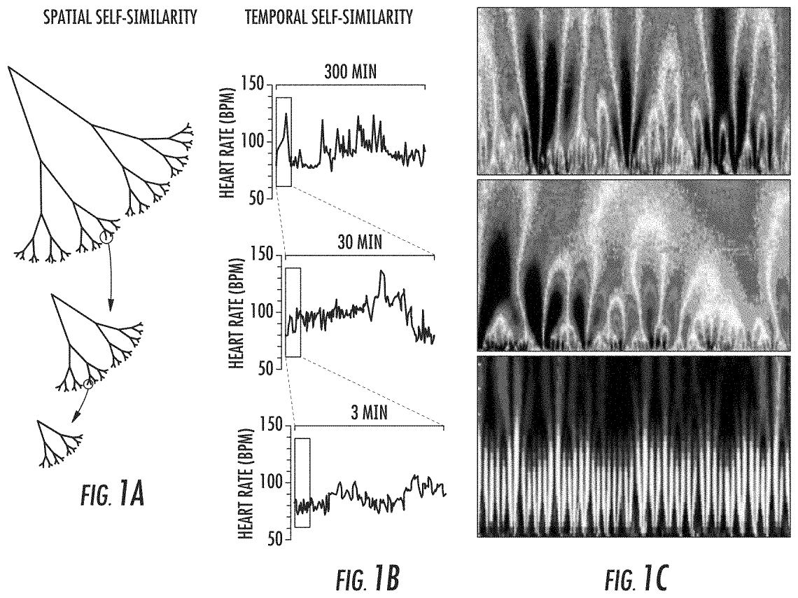

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In accordance with an aspect of the present invention, a device and a method allows for the nonlinear assessment of health and mortality. In order to nonlinearly determine health and mortality, a ventricular repolarization interval (QT) time series from a subject is obtained for a temporal interval and a ventricular activation interval (RR) time series is obtained from the subject for the same temporal interval. The method includes first, calculating entropy in the QT time series over the temporal interval to determine health and mortality. The method also includes calculating additional entropy values over the same temporal interval for the RR and other time-varying physiological signals such as the temperature, blood pressure, respiration, saturation of peripheral oxygen, intracardiac pressures and electroencephalogram time series. Additionally, the method includes comparing the first QT entropy with the entropy values of the other physiological signals to determine health and mortality.

The present invention uses a calculation referred to herein as EntropyX, in order to nonlinearly determine health and mortality. EntropyX accounts for the dynamics of cardiac repolarization, i.e., the QT interval time series, accounts for the dynamics of ventricular activation, i.e., the RR interval time series, and accounts for the dynamics of other time-varying physiological signals. Further optimization to account for the degree of coupling and shared information between QT and other time-varying physiological signals including RR intervals has led to the development of several variants of EntropyX (i.e., equations 1-11) as well as comparisons between the different time series based on mutual information, total correlation and Kullback-Leibler divergence (also known as relative entropy) and cross entropy using varying degrees of tolerance for matching. For the latter, templates from the time series of one physiological signal are matched to the time series of another physiological signal, and vice versa.

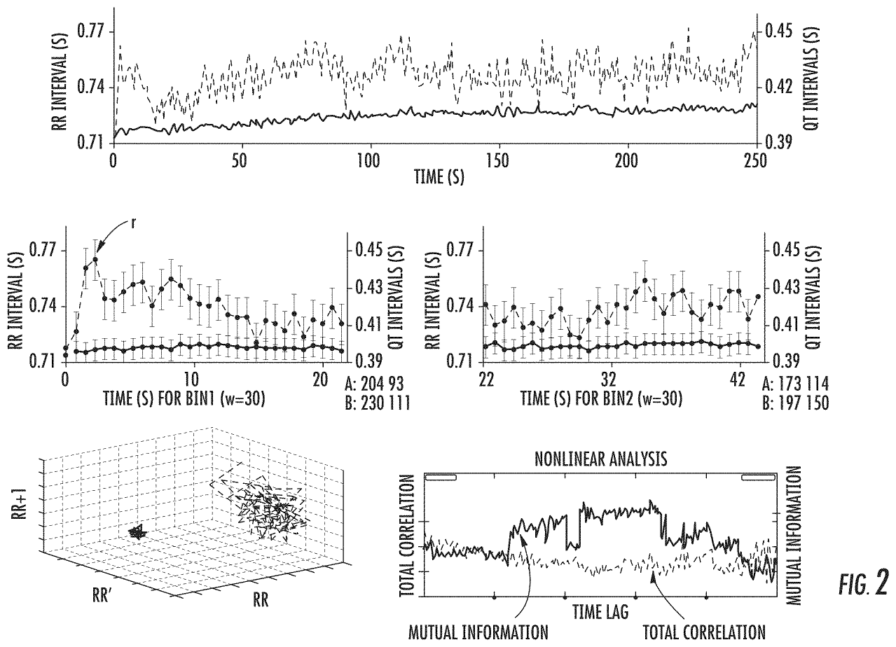

The variant of EntropyX involving mutual information nonlinearly quantifies the range of the probability density function (i.e., reduction in uncertainty) of the QT interval time series based on knowledge of the RR interval time series. Examples of various degrees of coupling information are shown in FIGS. 2-6.

EntropyX is conceptually simple, computationally straightforward and easily applicable in implantable devices, ambulatory settings and telemetry monitors. Novel features of EntropyX include nonlinear quantification of the dynamics of cardiac repolarization while ensuring confident probability estimates and interpreting quadratic entropy rate as a measure of Gaussian white noise, nonlinear quantification of the dynamics of cardiac repolarization in relation to the dynamics of other time varying physiological signals (e.g., accounting for hysteresis independent of any phase varying relationships between QT and RR intervals), and quantifying the degree of coupling and shared information between cardiac repolarization, ventricular activation and other physiological signals.

Unlike prior strategies such as SampEn or ApEn, EntropyX is insensitive to both the degree of tolerance allowed for matching templates and to the presence of outlying points. Unlike ApEn, frequency domain measures or geometric measures such as Poincaré plots, EntropyX is accurate in short time series. EntropyX is distinct from COSEn in the following ways:
1. EntropyX was optimized specifically for predicting mortality risk whereas COSEn was designed specifically for detection of atrial fibrillation
2. EntropyX uses a higher embedding dimension or template length (m)≥3 whereas COSEn is defined using m=1
3. COSEn was optimized to use very short records of RR intervals (i.e., N≤12 intervals), but EntropyX was optimized for analysis of a higher number of sampled intervals (i.e., N≥20 intervals)
4. Whereas COSEn is normalized for the matching volume [i.e., for tolerance r and template length m, the matching volume is (2×r)^{m}], but EntropyX is normalized for the matching area, i.e., (2×r).
5. Whereas the calculation of COSEn requires normalizing of the sample entropy for the heart rate, EntropyX does not require this normalization and functions independent of the heart rate information.
6. Whereas COSEn was designed specifically for analysis of RR intervals, EntropyX is not limited to analysis of the RR intervals and was optimized for quantifying the dynamics of the QT interval, respiration, blood pressure, temperature, intracardiac pressures, saturation of peripheral oxygen, and electroencephalogram time series.

Merely by way of example and not intended to be considered limiting, to illustrate how the algorithm works, suppose that for FIGS. 2-6, the sequence of 30 interval samples (i.e., N=30) for the RR and QT intervals shown in the two bins of the middle panel, of average quality recording at adequate sampling rate (i.e., R=1), the minimum number of matches is designated as n=15, and the template, m=3. The error bars represent the tolerance r.

In FIG. 2, the bin on the left of the middle panel, the number of matches in the QT interval at m=3 is 230 and at m=4 is 204, and the optimal value of r for n=15 is 2.5 msec. Thus, using equation 5,

$$E n t r o p y {X}_{Q T} = [ - ln \frac{204}{230} + ln ( 2 \times 2.5 ) ] = - ln ( 0.89 ) + ln ( 5 ) = 1.73$$

Similarly, using equation 6,

$$E n t r o p y {X}_{R R} = [ - ln \frac{93}{111} + ln ( 2 \times 12.5 ) ] = - ln ( 0.84 ) + ln ( 25 ) = 3.40$$

and using equation 7,

EntropyX_{RRQT1}=EntropyX_{RR}−EntropyX_{QT}=3.40−1.73=1.67

In the bin on the right of the middle panel, the number of matches in the QT interval at m=3 is 173 and at m=4 is 197, and the optimal value of r for n=15 is 2.5 msec. Thus,

$${EntropyX}_{Q T} = [ - ln \frac{173}{197} + ln ( 2 \times 2.5 ) ] = - ln ( 0.88 ) + ln ( 5 ) = 1.74$$

$${EntropyX}_{R R} = [ - ln \frac{114}{150} + ln ( 2 \times 12.5 ) ] = - ln ( 0.76 ) + ln ( 25 ) = 3.49$$

$${EntropyX}_{R R Q T 1} = {EntropyX}_{R R} - {EntropyX}_{Q T} = 3.49 - 1.74 = 1.75$$

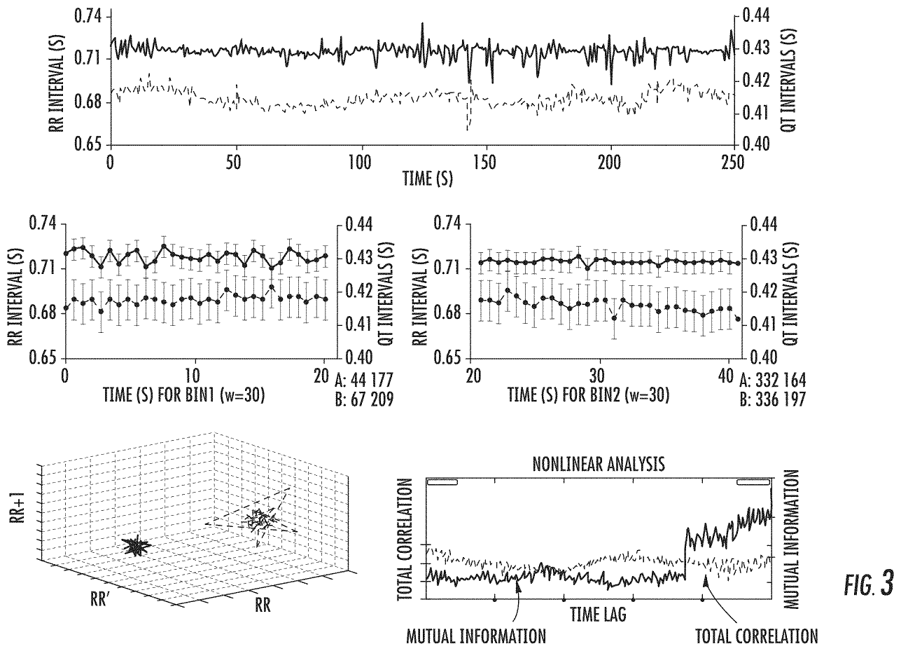

In FIG. 3, the bin on the left of the middle panel, the number of matches in the QT interval at m=3 is 44 and at m=4 is 67, and the optimal value of r for n=15 is 2.5 msec. Thus,

$${EntropyX}_{Q T} = [ - ln \frac{44}{67} + ln ( 2 \times 2.5 ) ] = - ln ( 0.66 ) + ln ( 5 ) = 2.03$$

$${EntropyX}_{R R} = [ - ln \frac{177}{209} + ln ( 2 \times 7.5 ) ] = - ln ( 0.85 ) + ln ( 15 ) = 2.87$$

$${EntropyX}_{R R Q T 1} = {EntropyX}_{R R} - {EntropyX}_{Q T} = 2.87 - 2.03 = 0.84$$

In the bin on the right of the middle panel, the number of matches in the QT interval at m=3 is 332 and at m=4 is 336, and the optimal value of r for n=15 is 2.5 msec. Thus,

$${EntropyX}_{Q T} = [ - ln \frac{332}{336} + ln ( 2 \times 2.5 ) ] = - ln ( 0.99 ) + ln ( 5 ) = 1.62$$

$${EntropyX}_{R R} = [ - ln \frac{164}{197} + ln ( 2 \times 7.5 ) ] = - ln ( 0.83 ) + ln ( 15 ) = 2.89$$

$${EntropyX}_{R R Q T 1} = {EntropyX}_{R R} - {EntropyX}_{Q T} = 2.89 - 1.62 = 1.27$$

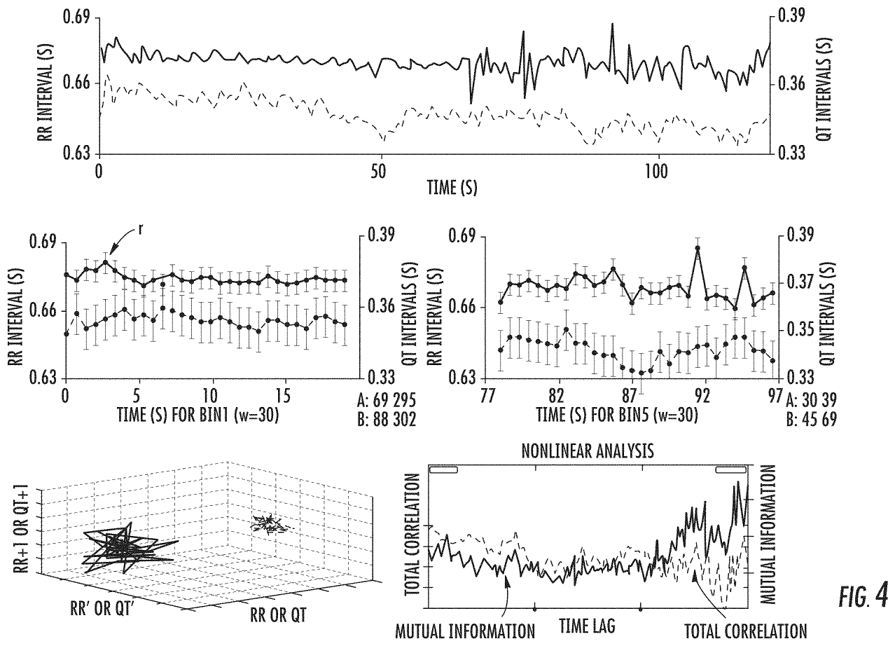

In FIG. 4, the bin on the left of the middle panel, the number of matches in the QT interval at m=3 is 69 and at m=4 is 88, and the optimal value of r for n=15 is 2.5 msec. Thus,

$${EntropyX}_{Q T} = [ - ln \frac{69}{88} + ln ( 2 \times 2.5 ) ] = - ln ( 0.78 ) + ln ( 5 ) = 1.85$$

$${EntropyX}_{R R} = [ - ln \frac{295}{302} + ln ( 2 \times 7.5 ) ] = - ln ( 0.98 ) + ln ( 15 ) = 2.73$$

$${EntropyX}_{R R Q T 1} = {EntropyX}_{R R} - {EntropyX}_{Q T} = 2.73 - 1.85 = 0.88$$

In the bin on the right of the middle panel, the number of matches in the QT interval at m=3 is 199 and at m=4 is 225, and the optimal value of r for n=15 is 2.5 msec. Thus,

$${EntropyX}_{Q T} = [ - ln \frac{199}{225} + ln ( 2 \times 2.5 ) ] = - ln ( 0.88 ) + ln ( 5 ) = 1.73$$

$${EntropyX}_{R R} = [ - ln \frac{244}{269} + ln ( 2 \times 7.5 ) ] = - ln ( 0.91 ) + ln ( 15 ) = 2.81$$

$${EntropyX}_{R R Q T 1} = {EntropyX}_{R R} - {EntropyX}_{Q T} = 2.81 - 1.73 = 1.07$$

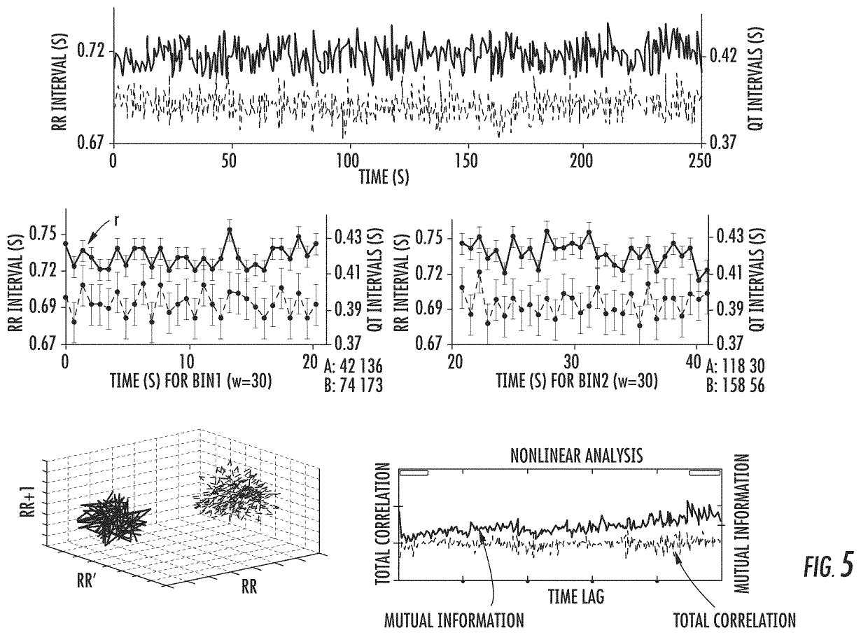

In FIG. 5, the bin on the left of the middle panel, the number of matches in the QT interval at m=3 is 42 and at m=4 is 74, and the optimal value of r for n=15 is 2.5 msec. Thus,

$${EntropyX}_{Q T} = [ - ln \frac{42}{74} + ln ( 2 \times 7.5 ) ] = - ln ( 0.57 ) + ln ( 15 ) = 3.27$$

$${EntropyX}_{R R} = [ - ln \frac{136}{173} + ln ( 2 \times 12.5 ) ] = - ln ( 0.79 ) + ln ( 25 ) = 3.46$$

EntropyX_{RRQT1}=EntropyX_{RR}−EntropyX_{QT}=3.46−3.27=0.19

In the bin on the right of the middle panel, the number of matches in the QT interval at m=3 is 118 and at m=4 is 158, and the optimal value of r for n=15 is 12.5 msec. Thus,

$${EntropyX}_{Q T} = [ - ln \frac{118}{158} + ln ( 2 \times 12.5 ) ] = - ln ( 0.75 ) + ln ( 25 ) = 3.51$$

$${EntropyX}_{R R} = [ - ln \frac{30}{56} + ln ( 2 \times 7.5 ) ] = - ln ( 0.54 ) + ln ( 15 ) = 3.33$$

$${EntropyX}_{R R Q T 1} = {EntropyX}_{R R} - {EntropyX}_{Q T} = 3.33 - 3.51 = - 0.18$$

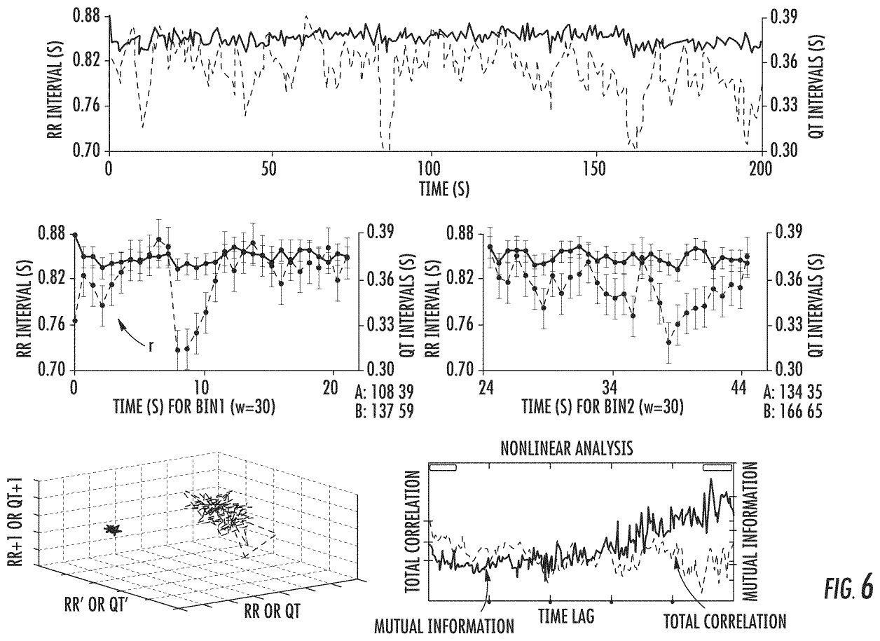

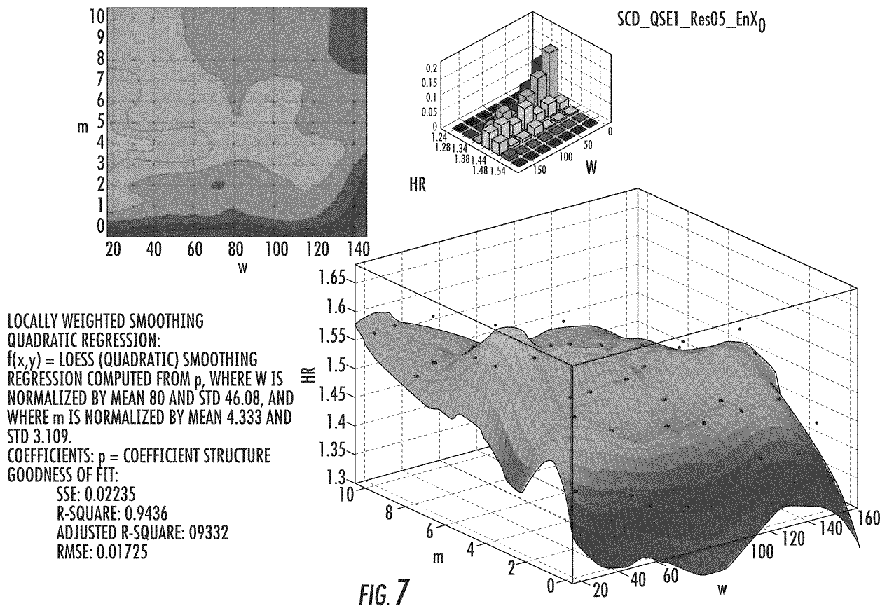

In FIG. 6, the bin on the left of the middle panel, the number of matches in the QT interval at m=3 is 108 and at m=4 is 137, and the optimal value of r for n=15 is 7.5 msec. Thus,

$${EntropyX}_{Q T} = [ - ln \frac{108}{137} + ln ( 2 \times 7.5 ) ] = - ln ( 0.57 ) + ln ( 15 ) = 2.95$$

$${EntropyX}_{R R} = [ - ln \frac{39}{59} + ln ( 2 \times 27.5 ) ] = - ln ( 0.66 ) + ln ( 55 ) = 4.42$$

$${EntropyX}_{R R Q T 1} = {EntropyX}_{R R} - {EntropyX}_{Q T} = 4.42 - 2.95 = 1.48$$

In the bin on the right of the middle panel, the number of matches in the QT interval at m=3 is 134 and at m=4 is 166, and the optimal value of r for n=15 is 7.5 msec. Thus,

$${EntropyX}_{Q T} = [ - ln \frac{134}{166} + ln ( 2 \times 7.5 ) ] = - ln ( 0.75 ) + ln ( 15 ) = 2.92$$

$${EntropyX}_{R R} = [ - ln \frac{35}{65} + ln ( 2 \times 27.5 ) ] = - ln ( 0.54 ) + ln ( 55 ) = 4.63$$

$${EntropyX}_{R R Q T 1} = {EntropyX}_{R R} - {EntropyX}_{Q T} = 4.63 - 2.92 = 1.70$$

In addition to the equations listed above for calculating EntropyX, at least one metric for health and mortality selected from a group consisting of recurrence plot analyses, correlation dimension, fractal complexity, cross correlation and mutual information can also be used on the QT interval time series and other time varying physiological signals. The method can further include determining a treatment plan for the subject using a result of the equation.

The correlation dimension measures the complexity or “strangeness” of a time series, often referred to as a type of fractal dimension, and provides information on the minimum number of dynamic variables needed to model the underlying system. The distance function of the correlation dimension is defined as

$${u}_{j} = \sqrt{{\sum }_{l = 1}^{m} {[ {u}_{j} ( l ) - {u}_{k} ( l ) ]}^{2}}$$

The correlation dimension D₂ is defined by

$${D}_{2} ( m ) = {lim}_{r \to 0} {lim}_{N \to ∞} \frac{log {C}^{m} ( r )}{log r}$$

The value of D₂ can be approximated by the slope from the linear part of the regression curve of log C^{m}(r) and log r. With increasing values of m (i.e., m=10), D₂ reaches a finite saturating value.

The fractal complexity of a time series can also be analyzed by the Recurrence plot, using vectors

uⱼ=(RRⱼ,RR_{j+τ}, . . . ,RR_{j+(m-1)τ}),j=1,2, . . . N−(m+1)τ

where m is the embedding dimension and r is the embedding lag. The vectors uⱼ then represent the RR interval time series as a trajectory in m dimensional space. A recurrence plot is a symmetrical [N−(m+1)×τ]×[N−(m+1)×τ] matrix of zeros and ones. The element in the j'th row and k'th column of the RP matrix, i.e. RP(j,k)=1 if the point uⱼ on the trajectory is close to point uₖ.

$$RP ( j , k ) = \{ \begin{matrix}1 & d ( {u}_{j} , {u}_{k} ) \leq r \\ 0 & otherwise\end{matrix}$$

where d(uⱼ,uₖ) is the Euclidean distance and r is a fixed threshold. The structure of the RP matrix usually shows short line segments of ones parallel to the main diagonal.

The lengths of these diagonal lines describe the duration of which the two points are close to each other, and are directly related to the ratio of determinism or predictability inherent to the system.

The recurrence rate quantifies the RP matrix by a ratio of ones and zeroes in the RP matrix and calculated using m=10, τ=1, and r=\sqrt{m}×SD (where SD is the standard deviation of the interval time series).

$$Recurrence rate = \frac{1}{N - m + 1} {\sum }_{j , k = 1}^{N - m + 1} RP ( j , k )$$

Other RP measures include lengths of the diagonal lines, using a threshold (lₘᵢₙ=2) to exclude the diagonal lines formed by tangential motion of the trajectory. The divergence is the inverse of the maximum line length, and correlates with the largest positive Lyapunov exponent.

Suppose that the states at times j and k are neighbouring, i.e., RP(j,k)=1, and if the system behaves predictably, similar situations will lead to a similar future, i.e. that the probability for RP(j+1, k+1)=1 will be high.

For perfectly predictable systems (e.g., sine function), the diagonal lines will be infinitely long. In contrast, stochastic systems will have a small probability for RP(j+1, k+1)=1 and the RP will have only single points or short lines. Chaotic systems will initially have exponentially diverging neighbouring states. A faster divergence rate will have a higher Lyapunov exponent and shorter diagonals.

It should be noted that the calculations discussed above can be completed and the resultant metrics used to assess a subject's risk of mortality and morbidity. In this context the subject can be a human or non-human subject. Resultant metrics classified as being in a high risk group can then be identified by a physician, nurse, technician, or other patient care specialist, and the patient's treatment protocol can be adjusted accordingly. In many cases, the subject can be monitored more closely in order to detect any potentially life threatening episodes. Alternately, mitigating treatment or medication can also be given to the patient.

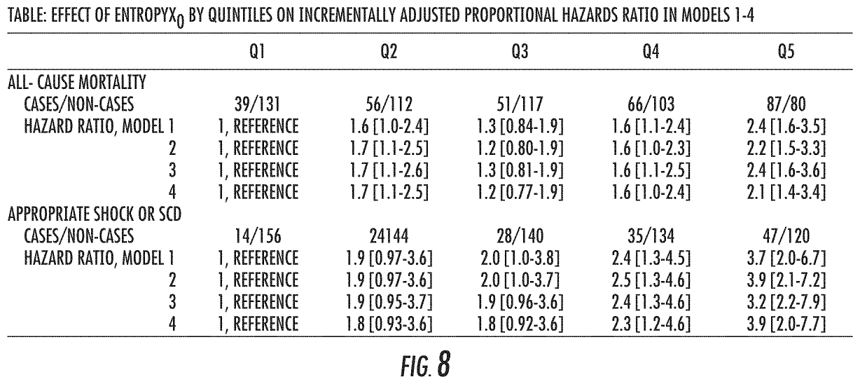

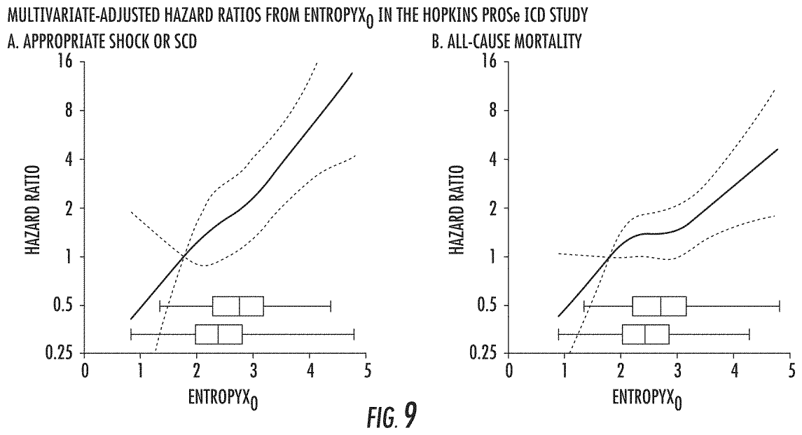

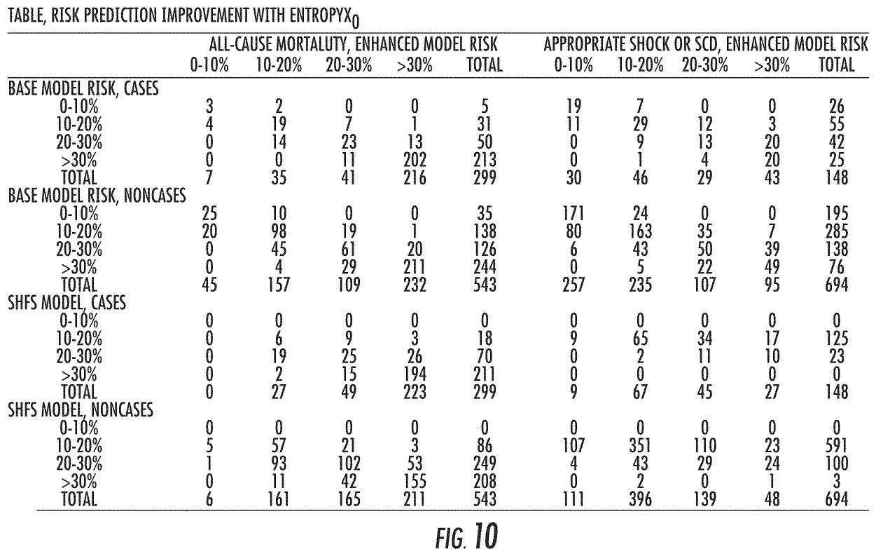

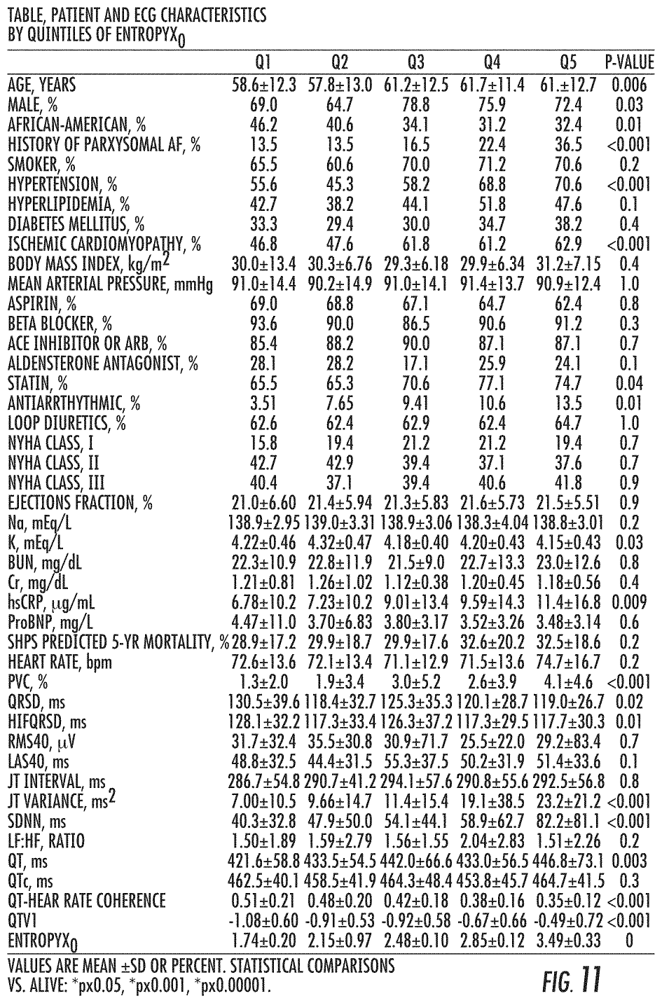

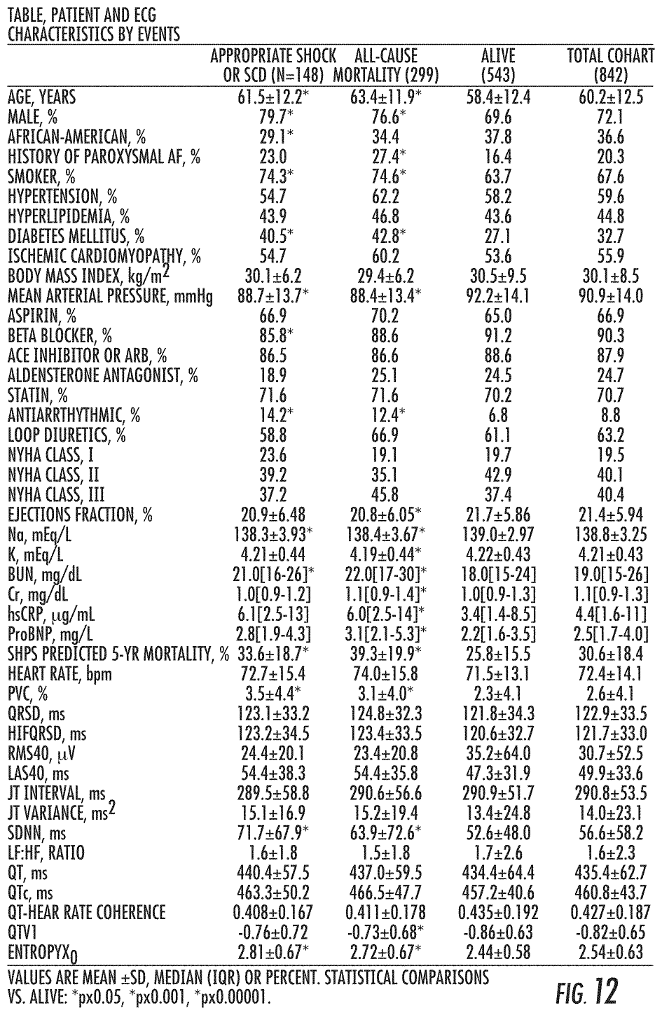

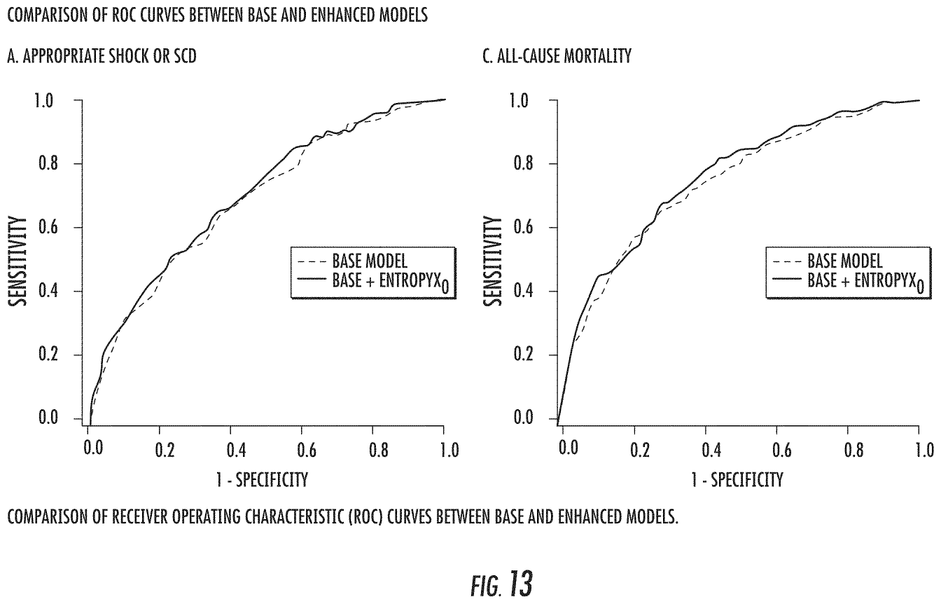

Examples of individual patients tested and monitored are included in FIGS. 2-6. Examples of summary results are included in FIGS. 8-15. These examples are included merely to illustrate the invention and are not meant to be considered limiting. The above described invention can be used in any way known to or conceivable by one of skill in the art.

FIGS. 8-13 illustrate a summary of preliminary results from heart failure patients in the Johns Hopkins PROSe ICD study in which EntropyX_{QT} was calculated from a 5 minute ECG collected at baseline. It should be noted that EntropyX_{QT} is among the most accurate indicators of mortality in the patients studied. EntropyX_{QT} was independently predictive of outcome above and beyond a comprehensive set of conventional predictors. EntropyX_{QT} had the same predictive value regardless of age, gender, race, ischemic cardiomyopathy or nonischemic cardiomyopathy, absence or presence of established risk factors and MRI parameters, including ejection fraction, left ventricular end diastolic pressure, and degree of fibrosis. This is the first report showing higher entropy of cardiac repolarization is strongly and independently associated with SCD and all-cause mortality.

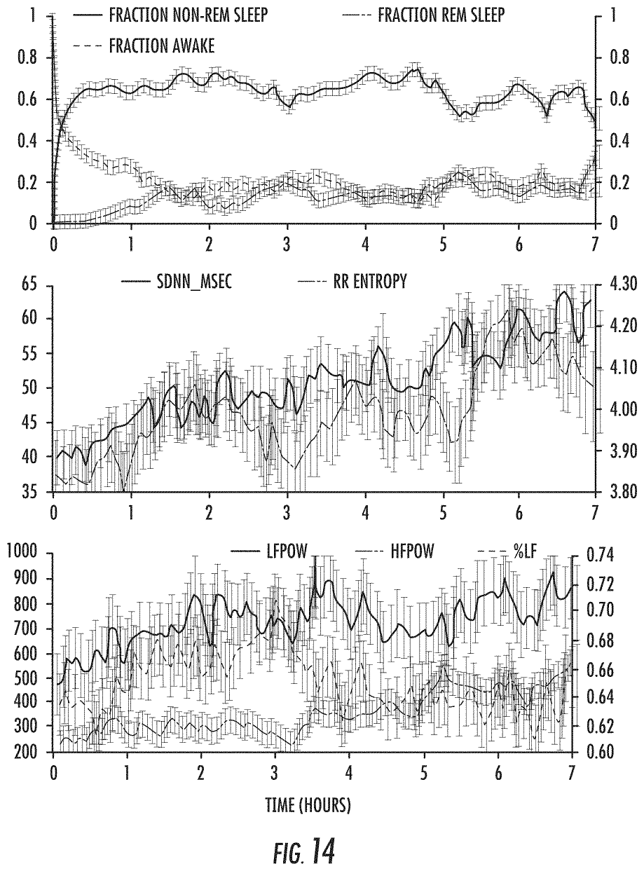

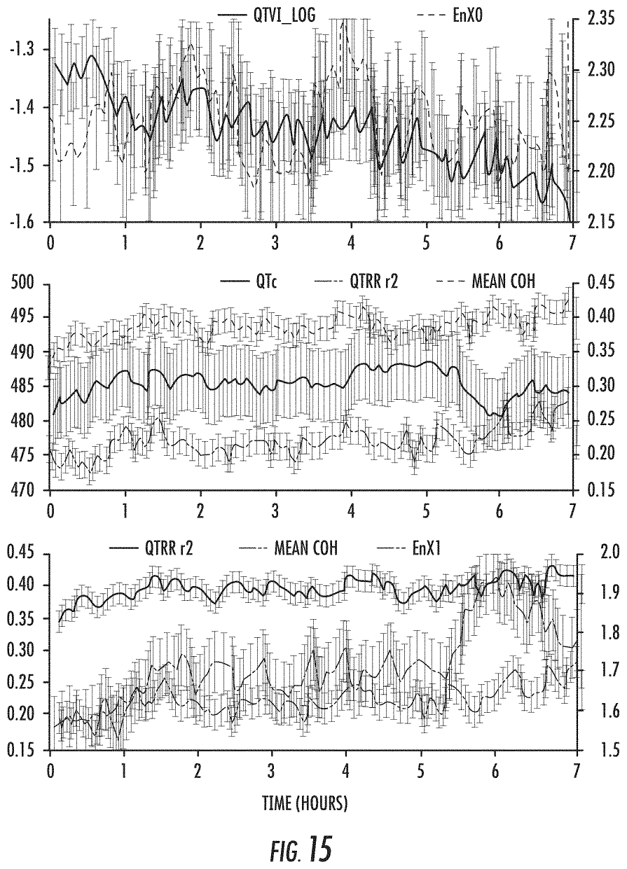

FIGS. 14-15 illustrate a summary of preliminary results from normal human subjects in the Sleep Heart Health Study. The plots show continuous overnight monitoring results for stages of sleep, heart rate variability (SDNN_msec), EntropyX_{RR} (also referred to as RR entropy), frequency domain analyses of low frequency power (LFPow), high frequency power (HFPow) and percent low frequency power (% LF). These results demonstrate that EntropyX_{QT}, EntropyX_{RR} and EntropyX_{RRQT1} measure physiological changes that are distinct from each other and from conventional measures of variability. Furthermore, the values of EntropyX_{QT} in these normal subjects are significantly lower than those in heart failure patients in the PROSe ICD study.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method of nonlinearly determining health and mortality for a subject comprising:
obtaining one or more series of cardiac repolarization (QT) time intervals for the subject;
calculating QT entropy for the subject using an EntropyX dynamic nonlinear analysis method; and
producing an output to a medical care provider to predict a patient's clinical prognosis for heart conditions.

2. The method of claim 1 further comprising:
obtaining physiological time series data from the subject;
calculating the entropy for each of the physiological time series for the subject using the EntropyX dynamic nonlinear analysis method;
comparing the QT entropy with the entropies of each of the physiological time series; and
producing an output to a medical care provider to predict a patient's clinical prognosis for heart conditions.

3. The method of claim 2 wherein the physiological time series data further comprises one chosen from a group consisting of an RR interval time series, temperature, blood pressure, respiration, saturation of peripheral oxygen, intracardiac pressures and electroencephalogram.

4. The method of claim 3 further comprising simultaneously obtaining the one or more series of QT time intervals and the RR interval time series.

5. The method of claim 4 wherein the physiological time series data are gathered using one selected from a group consisting of surface electrocardiogram, telemetry monitor, and intracardiac electrocardiogram (EGM) waveforms.

6. The method of claim 1 further comprising grouping the one or more series of QT time intervals into a plurality of subsets of the series of intervals wherein each subset consists of 20 or more intervals.

7. The method of claim 1 further comprising determining numbers of matching intervals within each one of a segment making up the one or more series of QT time intervals and using a regression model to combine the numbers of matching QT time intervals.

8. The method of claim 1 further comprising, using moment statistics as well as an additional method of nonlinear analysis, wherein the additional method of nonlinear analysis comprises one selected from a group consisting of-recurrence plot analyses, correlation dimension, fractal complexity, cross entropy, mutual information and cross correlation.

9. The method of claim 1 further comprising averaging entropy data from each of a plurality of subsets of the one or more series of QT time intervals.

10. The method of claim 3 further comprising calculating an absolute entropy from the RR interval time series.

11. The method of claim 1 further comprising calculating and absolute entropy from the one or more series of QT time intervals.

12. The method of claim 2 further comprising calculating absolute entropy values.

13. The method of claim 2 further comprising calculating a relative QT interval entropy of the one or more series of QT time intervals based on comparison between the QT entropy and a corresponding entropy of one of the physiological time series, and by matching interval data from the one of the physiological time series to QT interval data within the same physiological time series.

14. The method of claim 13 further comprising calculating changes in the relative QT interval entropy of the one or more series of QT time intervals over a time scale ranging from seconds to a number of years, wherein the number of years is less than or equal to a lifetime of the subject.

15. The method of claim 11 further comprising calculating changes in absolute QT entropy of the one or more series of QT time intervals over a time scale ranging from seconds to a number of years, wherein the number of years is less than or equal to a lifetime of the subject.

16. The method of claim 10 further comprising calculating changes in the absolute RR entropy over a time scale ranging from seconds to a number of years, wherein the number of years is less than or equal to a lifetime of the subject.

17. The method of claim 2 further comprising calculating changes in entropy of other physiological time series over a time scale ranging from seconds to a number of years, wherein the number of years is less than or equal to a lifetime of the subject.

18. The method of claim 13 further comprising calculating EntropyX by comparing the relative QT interval entropy with a relative entropy of one of the physiological time series over a time scale ranging from seconds to days.

19. The method of claim 1 further comprising calculating changes in EntropyX over a time scale ranging from seconds to a number of years, wherein the number of years is less than or equal to a lifetime of the subject.

20. The method of claim 1 further comprising generating a risk score from EntropyX.

21. The method of claim 3 further comprising calculating the entropy of the QT interval, entropy of the RR interval time series and entropies of the physiological time series data with an equation comprising one selected from the group consisting of
$${EntropyX}_{Y} ( m , N , n , R ) = {ExtropyX}_{α} - [ - ln \frac{{C}_{Y}^{m + 1} ( {r}_{Y} )}{{C}_{Y}^{m} ( {r}_{Y} )} + ln ( 2 \times {r}_{Y} ) ] , \\ {Entropy X}_{Q T} ( m , N , n , R ) = [ - ln \frac{{C}_{Q T}^{m + 1} ( {r}_{Q T} )}{{C}_{Q T}^{m} ( {r}_{Q T} )} + ln ( 2 \times {r}_{Q T} ) ] , and$$
$${EntropyX}_{R R} ( m , N , n , R ) = [ - ln \frac{{C}_{R R}^{m + 1} ( {r}_{Q T} )}{{C}_{R R}^{m} ( {r}_{Q T} )} + ln ( 2 \times {r}_{R R} ) ] ,$$
where γ is a physiological signal, m is an embedding dimension or template length, N is a number of sampled intervals per bin of the time series, n is a number of matches, r is a calculated tolerance for a given N that satisfies the specified n but without perfect matches, R is a specified precision of the data from which the initial value of the tolerance r is designated for subsequent iterative calculations, C_{Y}^{m}(r_{Y}) is a total number of matches within r of length m in the Y time series, C_{Y}^{m+1}(r_{Y}) represents a total number of matches within r of length m+1 in the Y time series, and EntropyX_{α} is an entropy of the time series of another physiological signal such as the QT interval, RR interval, temperature, blood pressure, respiration, intracardiac pressures, saturation of peripheral oxygen or electroencephalogram time series, QT is the QT interval and RR is the RR interval.

22. The method of claim 2 further comprising nonlinearly comparing the dynamics of two or more of the physiological time series consisting of N number of intervals with an equation comprising one selected from the group consisting of:
$${EntropyX}_{α Y 1} ( {m}_{α} , {m}_{Y} , {r}_{α} , {r}_{Y} , N , n , {R}_{α} , {R}_{Y} ) = [ - ln \frac{{C}^{m + 1} ( {r}_{α} )}{{C}^{m} ( {r}_{α} )} + ln ( 2 \times {r}_{α} ) ] - [ - ln \frac{{C}^{m + 1} ( {r}_{Y} )}{{C}^{m} ( {r}_{Y} )} + ln ( 2 \times {r}_{Y} ) ] , \\ {EntropyX}_{αY2} ( {m}_{α} , {m}_{Y} , {r}_{α} , {r}_{Y} , N , n , {R}_{α} , {R}_{Y} ) = - \frac{{C}^{m + 1} ( {r}_{α} )}{{C}^{m} ( {r}_{α} )} \times ln \frac{{C}^{m + 1} ( {r}_{Y} )}{{C}^{m} ( {r}_{Y} )} + ln [ {r}_{Y} + {r}_{α} ] , \\ {EntropyX}_{α Y 3} ( {m}_{α} , {m}_{Y} , {r}_{α} , {r}_{Y} , N , n , {R}_{α} , {R}_{Y} ) = - ln \frac{{C}^{m + 1} ( {r}_{α} )}{{C}^{m} ( {r}_{Y} )} , \\ {Entropy X}_{α Y 4} ( {m}_{α} , {m}_{Y} , {r}_{α} , {r}_{Y} , N , n , {R}_{α} , {R}_{Y} ) = - ln \frac{{C}^{m + 1} ( {r}_{Y} )}{{C}^{m} ( {r}_{α} )} , and \\ {Entropy X}_{α Y 5} ( {m}_{α} , {m}_{Y} , {r}_{α} , {r}_{Y} , N , n , {R}_{α} , {R}_{Y} ) = [ - ln \frac{{C}^{m + 1} ( {r}_{Y} )}{{C}^{m} ( {r}_{α} )} ] - [ - ln \frac{{C}^{m + 1} ( {r}_{α} )}{{C}^{m} ( {r}_{Y} )} ] + ln [ {r}_{Y} + {r}_{α} ]$$
where r is a tolerance, α and Y are two time series, N is the number of intervals, n is a number of matches, C_{Yα}^{m}(r_{Y}) is defined as a total number of matches in the Y time series within r_{Y} of templates formed in the α time series within r_{α} of length m, CY (r_{Y}) is defined as a total number of matches in the Y time series within r_{Y} of templates formed in the α time series within r_{α} of length m+1, C_{αY}^{m}(r_{α}) is defined as a total number of matches in the α time series within r_{α} of templates formed in the Y time series within r_{Y} of length m, and C_{αY}^{m+1}(r_{α}) is defined as a total number of matches in the α time series within r_{α} of templates formed in the Y time series within r_{Y} of length m+1.

23. The method of claim 1 wherein the subject further comprising one selected from the group consisting of humans, primates, dogs, horses, guinea pigs, cats, and fruit flies.

24. The method of claim 1 further comprising the method being performed using one selected from a group consisting of a computer, a computer readable medium, a server, a processing device, a cellular phone, and a tablet computing device.
